(19) United States
(12) Patent Application Publication
(10) Pub. No.: US 2022/0346723 A1
(43) Pub. Date: Nov. 3, 2022

(54) PREDICTION OF THE LONG-TERM HEDONIC RESPONSE TO A SENSORY STIMULUS

(71) Applicant: SYMRISE AG, Holzminden (DE)

(72) Inventors: Corey YEO, #14-14 Waterfront Gold (SG); Jonathan JACOBS, #03-03 Singapore (SG)

(21) Appl. No.: 17/282,506

(22) PCT Filed: Aug. 4, 2020
(86) PCT No.: PCT/EP2020/071885

§ 371 Date: Apr. 2, 2021

(30) Foreign Application Priority Data
Aug. 8, 2019 (SG) 10201907343S

(51) Int. Cl. A61B 5/00; A61B 5/16; A61B 5/0533; A61B 5/377

(52) U.S. Cl. CPC A61B 5/7275; A61B 5/163; A61B 5/0533; A61B 5/377

(57) ABSTRACT

A method of predicting the long-term hedonic response to at least one predetermined sensory stimulus for an individual is disclosed. The method comprises the steps of (a) exposing the individual to the at least one sensory stimulus for a number of times over an initial time period of exposure according to an exposure pattern, (b) for each exposure, obtaining data indicative of the individual's hedonic response to the at least one sensory stimulus, (c) providing the data indicative of the individual's hedonic response and the exposure pattern to a machine learning algorithm, and (d) predicting the individual's long-term hedonic response to the sensory stimulus by the machine learning algorithm for a time point a predetermined prediction time period after the initial time period of exposure.

### FIELD OF THE INVENTION

The present disclosure generally relates to a method of predicting the long-term hedonic response, such as the long-term liking, to at least one predetermined sensory stimulus, such as an olfactive stimulus, either for an individual or an audience.

### BACKGROUND OF THE INVENTION

An individual's first exposure to a given sensory stimulus may evoke a certain hedonic or emotional response. The same sensory stimulus, however, does not necessarily produce the same hedonic response on a subsequent exposure so that the initial hedonic response to a given sensory stimulus generally does not reliably ascertain the hedonic response to the same sensory stimulus at a later time. Rather, the hedonic response to a given sensory stimulus changes over time and upon repeated exposure. However, the evolution of the hedonic response over time is generally unknown in advance.

It is an object of the present invention to estimate the long-term hedonic response so that product development can focus on manufacturing consumer products with sensory stimuli having a hedonic response that will improve or at least not decline over time. In this way, time and energy spent on the development and eventually manufacturing of consumer products that have a considerable risk of not being put into use and finally end up on landfill can be reduced.

It is therefore an object of the present invention to provide a method allowing the streamlining of development of consumer products comprising at least one sensory stimulus, thereby avoiding waste and saving resources.

### SUMMARY OF THE INVENTION

The present invention is directed to a computer-implemented method of predicting the long-term hedonic response to at least one predetermined sensory stimulus for an individual, comprising (a) exposing the individual to the at least one sensory stimulus for a number of times over an initial time period of exposure according to an exposure pattern, (b) for each exposure, obtaining data indicative of the individual's hedonic response to the at least one sensory stimulus, (c) providing the data indicative of the individual's hedonic response and the exposure pattern to a machine learning algorithm, and (d) predicting the individual's long-term hedonic response to the sensory stimulus by the machine learning algorithm for a time point a predetermined prediction time period after the initial time period of exposure.

The method thus allows, using a machine learning algorithm, to predict an individual's hedonic response to a given sensory stimulus at a certain time after first being exposed to the given sensory stimulus. In a professional set up, the individual would be exposed to a certain sensory stimulus not just once, but a number of times during an initial time period of exposure. The number of exposures, the length of each exposure, and the time intervals between consecutive exposures during the initial time period of exposure determine the exposure pattern.

In certain embodiments, the individual is exposed at least twice, preferably three or more times to the sensory stimulus over a relatively short initial time period of exposure. For instance, the individual may be exposed to the sensory stimulus two or three times within one week. In other embodiments, the individual may, within a short time period, such as an hour, be subjected to a series of exposures to the sensory stimulus, such as three times, and this may be repeated a number of times, such as two or three times, within the initial time period of exposure, such as one week. The initial time period of exposure is preferably one week or less. The predetermined prediction time period is preferably three months or more.

When an individual was exposed to a sensory stimulus during an initial time period of exposure, the individual's long-term hedonic response to the same sensory stimulus is the individual's hedonic response to that sensory stimulus when a prediction time period measured from the end of the initial time period of exposure has lapsed. In one embodiment, an individual's hedonic response to a sensory stimulus is the individual's liking of that sensory stimulus. However, other measures of the long-term hedonic response are also possible.

The exposure pattern may have an influence on an individual's long-term hedonic response to a predetermined sensory stimulus. Therefore, when training the machine learning algorithm, training data with varying numbers of exposures, intervals during subsequent exposures, and initial time period of exposure should be provided.

The at least one sensory stimulus may be selected from a group comprising olfactive, auditory, haptic, taste and visual stimuli. The individual may be exposed to only a single sensory stimulus or to two or more sensory stimuli which may address the same sense or different senses. For instance, an individual may be exposed to an olfactive stimulus without any further sensory stimuli. If the individual's hedonic response is to be tested for two or more sensory stimuli, the two or more sensory stimuli are presented to each individual at the same time so that each exposure can be associated with not just one sensory stimulus, but the concurrence of the two or more sensory stimuli. For instance, an individual may be simultaneously exposed to an olfactive stimulus and a visual stimulus. Another combination of sensory stimuli an individual may be exposed to is a taste stimulus together with a visual stimulus. Other combinations of sensory stimuli are also possible. When reference is made to a sensory stimulus, this should be understood as also relating to a combination of a plurality of sensory stimuli, unless inappropriate.

The individual's hedonic response may be determined by obtaining the individual's psychological response to the at least one sensory stimulus. Preferably, the individual's psychological response is combined with data on the individual's psychometric response to the sensory stimulus.

The data indicative of the hedonic response to the at least one sensory stimulus for the individual are fed into a machine learning algorithm. Preferably, the machine learning algorithm was previously trained, cross-validated and tested using an extensive set of data collected previously across a large number of individuals and instances of sensory stimuli. The machine learning algorithm may produce predictions at the level of the individual for a given sensory stimulus.

The present invention is also directed to a computer-implemented method of predicting the long-term hedonic response of an audience to at least one predetermined sensory stimulus. The above described method performed on the level of an individual is repeated for a number of different individuals representative of a large audience, such as a consumer base. The predictions on the long-term hedonic response of each individual are then combined to predict the long-term hedonic response of the audience.

The present invention further pertains to a data processing apparatus comprising means for carrying out the above methods, to a computer program comprising instructions which, when the program is executed by a computer, cause the computer to carry the above methods, and a computer-readable medium comprising instructions which, when executed by a computer, cause the computer to carry out the above methods.

The present invention is further directed to a method of devising a consumer product, the method comprising the step of performing the above computer-implemented method of predicting the long-term hedonic response of an individual or an audience to at least one predetermined sensory stimulus for a plurality of sensory stimuli and determining a sensory stimulus of the plurality of sensory stimuli that has the maximum long-term hedonic response, with the method further comprising the step of devising a consumer product comprising the determined sensory stimulus. The sensory stimulus having the maximum long-term hedonic response from the plurality of sensory stimuli may be determined or confirmed using statistical methods, such as a standard significance test. The maximum long-term hedonic response can be the long-term hedonic response having the highest value indicating the strongest positive emotional response to a certain sensory stimulus.

The present invention further relates to a system configured to predict the long-term hedonic response to at least one predetermined sensory stimulus for an individual. The system comprises a measurement unit configured to measure, for each exposure of an individual to at least one sensory stimulus for a number of times over an initial time period of exposure according to an exposure pattern, data indicative of the individual's hedonic response to the at least one sensory stimulus, and a control unit configured to predict, based on the data indicative of the individual's hedonic response measured by the measurement unit and the exposure pattern, the individual's long-term hedonic response to the sensory stimulus by a machine learning algorithm for a time point a predetermined prediction time period after the initial time period of exposure. The measurement unit can comprise at least one of an EEG (electroencephalography) measurement device, GSR (galvanic skin response) measurement device, an eye-tracker, and a video recording device. An EEG measurement device is generally configured to measure, preferably non-invasively, electrical activity of an individual's brain. Other measurement devices for measuring brain activity can also be used, with one example being fMRI (functional magnetic resonance imaging). A GSR measurement device is generally configured to measure skin conductance of an individual. An eye-tracker is generally configured to measure at least one of eye position, eye movement, point of gaze, and pupil dilation and contraction of an individual.

### BRIEF DESCRIPTION OF THE DRAWINGS

FIG. 1 shows a flowchart illustrating a method according to the present invention.

FIG. 2 shows a flowchart illustrating a method to train, cross-validate and test a machine learning algorithm used in the method according to the present invention.

FIG. 3 shows a system for predicting the long-term hedonic response to at least one sensory stimulus for an individual or an audience.

FIG. 4 shows a raw EEG signal recorded from one electrode over 0.5 seconds.

FIG. 5 shows a power spectrum of EEG data for one electrode computed using a Fast

Fourier Transform over a 2 second time window, with the Hann window function.

FIG. 6 shows a GSR recording over 15 seconds.

### DETAILED DESCRIPTION OF THE INVENTION

Computer-implemented methods of predicting the long-term hedonic response to at least one predetermined sensory stimulus are described. Specifically, the methods described herein include predicting an individual's long-term hedonic response to at least one predetermined sensory stimulus, and predicting an audience's long-term hedonic response to at least one predetermined sensory stimulus. An audience comprises a large number of individuals.

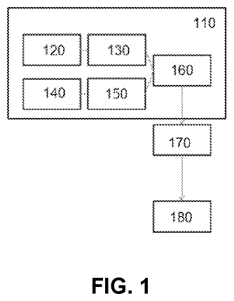

A method of predicting the long-term hedonic response to at least one predetermined sensory stimulus for an individual is shown as a flowchart in FIG. 1. The method involves exposing an individual to a predetermined sensory stimulus for the first time during an initial time period of exposure. The individual may be exposed to the sensory stimulus only once such that the initial time period of exposure has the length of the single exposure. To achieve more reliable predictions, the individual is preferably repeatedly exposed to the sensory stimulus over the course of the initial time period of exposure. For example, the individual is exposed to the sensory stimulus three times over one week. The individual's physiological responses, such as from EEG, GSR, eye-tracking, and/or video of face, are measured during each exposure. Additionally or alternatively, the individual's psychometric responses, such as from self-reports and/or response-time tests, are measured during each exposure. After noise removal from the physiological data as well as calibration and cleaning of the psychometric data, salient features are extracted from the data. The extracted salient features are then used as input into an artificial neural network which, when properly trained, cross-validated and tested, yields a prediction about the long-term hedonic response to the predetermined sensory stimulus for the individual. The predicted long-term hedonic response is generally expressed as a value within a range from minus infinity to plus infinity, with a value below 0 meaning a negative hedonic response, a value of 0 meaning indifference, and a value above 0 meaning a positive hedonic response to a certain sensory stimulus.

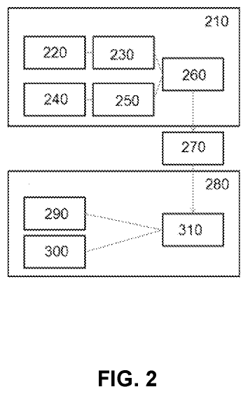

FIG. 2 shows a flowchart illustrating a method of training, cross-validating and testing an artificial neural network to be used in the method of FIG. 1. The present invention is not limited to neural networks and other machine learning algorithms may be used. Training data is obtained by exposing an individual to a predetermined sensory stimulus during an initial time period of exposure. The individual's physiological and/or psychometric responses are measured during each exposure. The psychological measurement data is freed from noise so that salient features can be extracted. Similarly, salient features are extracted from the psychometric measurement data after calibration and data cleaning. These steps are repeated for a large number of individuals, with varying lengths of the initial time period of exposure and exposure patterns as well as for various sensory stimuli. The extracted salient features from each initial time period of exposure (relating to a specific individual and a specific sensory stimulus or combination of sensory stimuli) along with the corresponding pattern of exposure are input into the artificial neural network for training purposes.

After a predetermined prediction time period, which preferably is no less than 3 months after the initial time period of exposure, further data are collected for each previously tested individual. In this way, data on long-term emotional response are obtained that can be used to train the machine learning algorithm. For instance, for each individual, the actual purchase or re-purchase behaviour of a consumer product having the sensory stimulus or combination of sensory stimuli used during the initial time period of exposure is observed. It is also possible to again expose each individual to the same sensory stimulus or combination of sensory stimuli as encountered during the initial time period of exposure and obtain data indicative of the hedonic response. Additionally or alternatively, self-reports of liking and/or self-reports of purchase intention of a consumer product that incorporates the sensory stimulus/stimuli and/or choice experiments including such a consumer product are conducted.

When the data on long-term hedonic response have not been collected in a uniform manner, i.e., different manifest variables were measured while trying to assess inherent latent variables relating to long-term hedonic response, in order to still be able to make use of these different types of measurement of long-term hedonic response for training purposes, they need to be harmonized. This is accomplished by forming latent variable models from studies where multiple modes of assessing long-term hedonic response were used together. These models provide a method of imputing latent variable scores from the measurements that were available in the training data.

Alternatively, to avoid the need for harmonization, data on long-term hedonic response could be collected in a uniform manner across respondents and these measurements used directly for training the machine learning algorithm.

During the training process, both the inputs (the extracted features obtained from step 260) and the desired outputs (long-term hedonic response obtained in step 310) are provided, and the artificial neural network processes the inputs and compares the resulting outputs against the desired outputs. After the training process is considered complete, some of the data obtained during steps 220, 240, 290 and 300 are used for testing the trained artificial neural network. The artificial neural network is also cross-validated, as is known by those skilled in the art.

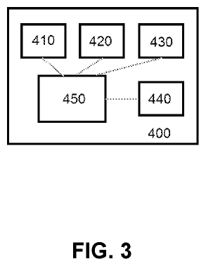

Psychological data collection using a system 400 as shown in FIG. 3 and feature extraction from these data is explained in more detail below.

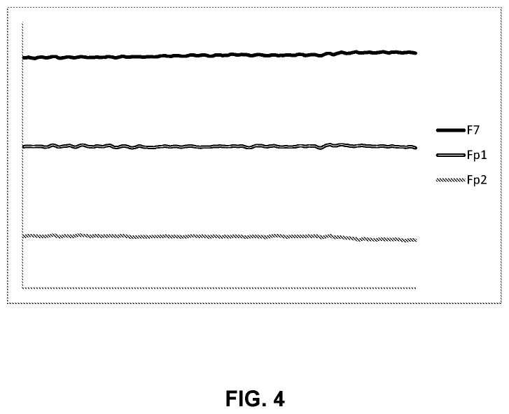

Raw EEG signals are collected using an EEG signal measuring device 410. In one embodiment, the EEG signal measuring device 410 has 14 to 20 electrodes or channels, and a sample rate between 128 Hz and 500 Hz. Exemplary raw EEG signals recorded from three electrodes at positions F7, Fp1, and Fp2 over 0.5 seconds are shown in FIG. 4.

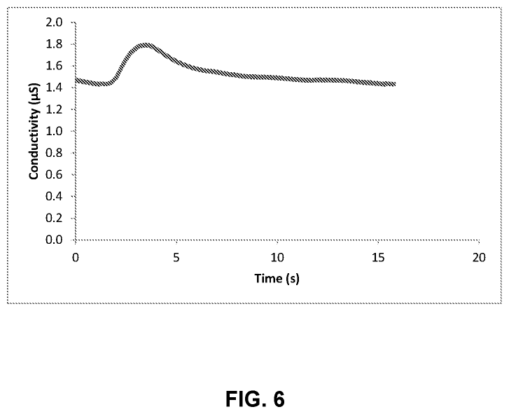

Additionally or alternatively, raw EDA (electrodermal activity) signals are collected using an EDA signal measuring device 420, such as a GSR signal measuring device. In one embodiment, the GSR signal measuring device 420 has one electrode or channel, and a sample rate between 5 Hz and 128 Hz. An exemplary raw GSR signal recorded over 15 seconds is shown in FIG. 6.

Additionally or alternatively, raw eye-tracking signals are collected using an eye tracker 430. In one embodiment, the eye-tracking signals are 2D Cartesian coordinates relative to a screen. The sample rate can be 30 Hz.

Additionally or alternatively, video signals of the individual, preferably the face, are captured using a video recording device 440, such as a webcam. The video signal can have various resolutions and sample rates.

When measuring the hedonic or emotional response to a sensory stimulus, it is important to know when exactly an individual begins to experience the sensory stimulus. Therefore, in one preferred embodiment, once the raw data signals have been collected, they are synchronized. While a visual stimulus can be controlled or timed easily as it is displayed on a screen, for an olfactive, taste or tactile stimulus, the present invention contemplates synchronizing a video signal of the individual with the other data signals, such as the EEG, GSR or eye-tracking signals. The data signals are synchronized by regularly checking the clock or timestamp of each data signal against the system clock of control unit 450 that is collecting the data. When any drift between the data signals occurs, adjustments can be made so as to eliminate the drift.

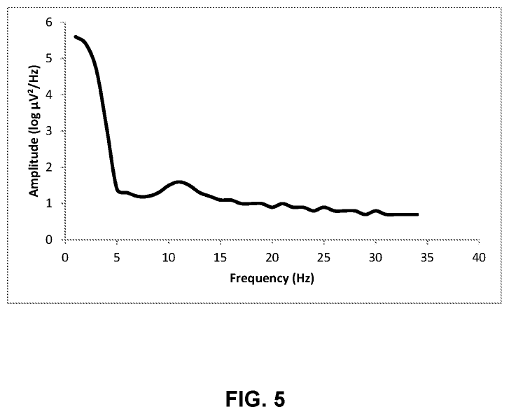

In one embodiment, during EEG signal processing, which can be performed by control unit 450, low-pass and high-pass filters are applied to the raw EEG signals so as to remove signal components with frequencies above 50 Hz and below 0.5 Hz. Thereafter, an Independent Component Analysis (ICA) is performed, and a machine learning algorithm matches the independent components against non-brain signals (e.g., eye blinks, head movement) and removes independent components identified as non-brain signals. For each channel, the EEG signal is then split into overlapping time windows. Each time window can be 2 seconds long, with a new time windows starting every 0.5 seconds. The Higuchi Fractal Dimension (HFD) is computed for each time window. Further, for each time window, a Hann window is applied and a Fast Fourier Transform (FFT) computed so as to obtain the EEG power spectrum. An exemplary EEG power spectrum at one electrode during a two second time window is shown in FIG. 5.

In one embodiment, during eye-tracking post-processing, which can be performed by control unit 450, the raw data is split into time windows representing the time that a slide image was continuously shown on a screen. For each window, the Cartesian coordinates are used to generate a heat map (a matrix with the same dimensions as the image on the screen) using Gaussian Kernel Estimation. The heat map can be plotted on top of the image as the final output.

In one embodiment, during GSR post-processing, which can be performed by control unit 450, continuous decomposition analysis is applied to decompose the raw GSR signal into phasic (fast moving) and tonic (slow moving) components. A peak detection algorithm can be used to determine from the raw GSR data a binary outcome at each point in time: whether the individual is in an emotionally aroused state or not.

The so processed measured data together with the exposure pattern, preferably expressed as a vector of time (step function), then serves as input to the machine learning algorithm on control unit 450 that outputs an estimated long-term hedonic response associated with the measured data.

An exemplary table of predicted outcomes for a fragrance A tested on a group of individuals is shown in Table 7. The average predicted long-term hedonic for the 16 individuals is 0.12 which means that the fragrance A is predicted to be received positively long-term. However, on average, the hedonic response is not very pronounced such that it may be decided that fragrance A may still need to be improved so as to achieve a better long-term hedonic response.

| | Predicted long-term |
|---|---|
| Individual | hedonic response |
| 1 | 0.27 |
| 2 | 0.18 |
| 3 | −0.10 |
| 4 | −0.23 |
| 5 | 0.41 |
| 6 | 0.21 |
| 7 | 0.83 |
| 8 | 0.08 |
| 9 | 0.99 |
| 10 | −0.29 |
| 11 | 0.44 |
| 12 | −0.36 |
| 13 | −0.50 |
| 14 | −0.65 |
| 15 | 0.21 |
| 16 | 0.47 |

1-12. (canceled)

13. A system for predicting a long-term hedonic response to at least one predetermined sensory stimulus for an individual, the system comprising:
a measurement unit that measures, for each exposure of an individual to at least one sensory stimulus for a number of times over an initial time period of exposure according to an exposure pattern, data indicative of the individual's hedonic response to the at least one sensory stimulus, and
a control unit that predicts the individual's long-term hedonic response to the sensory stimulus by a machine learning algorithm for a time point a predetermined prediction time period after the initial time period of exposure, based on the data indicative of the individual's hedonic response measured by the measurement unit and the exposure pattern.

14. The system of claim 13, wherein the control unit derives a long-term hedonic response to the at least one sensory stimulus of a group of individuals based on the long-term hedonic response of each individual to predict the long-term hedonic response of an audience, wherein a number of the individuals in the group of individuals is smaller than a number of individuals in the audience.

15. The system of claim 13, wherein the measurement unit comprises at least one of the group comprising an EEG measurement device, an GSR measurement device, an eye-tracker, and a video capturing device.

16. A method of predicting the long-term hedonic response to at least one predetermined sensory stimulus for an individual, comprising:
(a) exposing the individual to the at least one sensory stimulus for a number of times over an initial time period of exposure according to an exposure pattern,
(b) for each exposure, obtaining data indicative of the individual's hedonic response to the at least one sensory stimulus,
(c) providing the data indicative of the individual's hedonic response and the exposure pattern to a machine learning algorithm,
(d) predicting the individual's long-term hedonic response to the sensory stimulus by the machine learning algorithm for a time point a predetermined prediction time period after the initial time period of exposure.

17. The method of claim 16, wherein the individual's long-term hedonic response is the individual's long-term liking of the at least one predetermined sensory stimulus.

18. The method of claim 16, wherein the machine learning algorithm is based on an artificial neural network.

19. The method of claim 16, wherein the data indicative of the individual's hedonic response to the at least one sensory stimulus is obtained by measuring psychologic and/or psychometric responses of the individual.

20. The method of claim 16, wherein the data indicative of the individual's hedonic response to the at least one sensory stimulus comprises data relating to the individual's brain activity.

21. The method of claim 19, wherein the psychologic and/or psychometric responses of the individual are measured synchronously.

22. The method of claim 19, wherein measuring the psychologic response of the individual comprises measuring data obtained from a group comprising EEG, GSR, eye-tracking, and video of face.

23. The method of claim 16, wherein step (a) comprises exposing the individual to the at least one sensory stimulus at least three times.

24. The method of claim 16, wherein the predetermined prediction time period is at least 10-times longer than the initial time period of exposure.

25. The method of claim 16, wherein the at least one sensory stimulus is selected from a group comprising an olfactive, auditory, haptic, taste and visual stimulus.

26. A method of predicting the long-term hedonic response of an audience to at least one predetermined sensory stimulus using the method of claim 16, comprising:
repeating steps (a) to (d) for each individual of a group of individuals, wherein a number of the individuals in the group of individuals is smaller than a number of individuals in the audience, and
deriving a long-term hedonic response to the at least one sensory stimulus of the group of individuals based on the long-term hedonic response of each individual to predict the long-term hedonic response of the audience.

27. A method of devising a consumer product, the method comprising:
performing the method defined in claim 16 for a plurality of sensory stimuli,
determining from the plurality of sensory stimuli a sensory stimulus having the maximum long-term hedonic response, and
devising a consumer product comprising the determined sensory stimulus.
